(19) United States
(12) Patent Application Publication
(10) Pub. No.: US 2021/0221889 A1
(43) Pub. Date: Jul. 22, 2021

(54) USE OF A CHECKPOINT INHIBITOR IN COMBINATION WITH ULTRALOW DOSE WHOLE BODY IRRADIATION

(71) Applicant: ATOMIC ENERGY OF CANADA LIMITED/ÈNERGIE ATOMIQUE DU CANADA LIMITÈE, Chalk River (CA)

(72) Inventors: Yi WANG, Whitby (CA); Michelle BUGDEN, Pembroke (CA); Laura BANNISTER, Chalk River (CA); Dmitry KLOKOV, Chalk River (CA)

(21) Appl. No.: 15/734,890

(22) PCT Filed: Jun. 7, 2019
(86) PCT No.: PCT/CA2019/050810

§ 371 Date: Dec. 3, 2020

Related U.S. Application Data
(60) Provisional application No. 62/682,493, filed on Jun. 8, 2018.

(51) Int. Cl. C07K 16/28; A61N 5/10; A61P 35/00

(52) U.S. Cl. CPC C07K 16/2818; A61N 5/10; A61K 2039/505; A61P 35/00; A61N 2005/1085; C07K 16/2827

(57) ABSTRACT

The present invention relates to a method to improve the anti-tumour effects of immune checkpoint therapy by combining the use of a checkpoint inhibitor with ultralow dose whole body irradiation. The present invention enhances the anti-tumour effects of immune checkpoint therapy by reducing tumour volume as well as shortening the response time of the immune checkpoint therapy, as compared to immune checkpoint therapy on its own.

### CROSS-REFERENCE TO RELATED APPLICATION

The present application claims the benefit under 35 U.S.C. § 119(e) of provisional application Ser. No. 62/682,493, filed Jun. 8, 2018, the contents of which are hereby incorporated by reference.

### FIELD

In one of its aspects, the present disclosure relates generally to treating cancer, and more particularly to the prevention of tumour growth.

### BACKGROUND

Cancer is one of the leading causes of death worldwide, accounting for approximately 8 million deaths in 2015. Recently, immune checkpoint therapy, which can address tumour immune resistance by blocking inhibitory pathways within the immune system, has been used in cancer treatments. Both pre-clinical studies and clinical trials have demonstrated improved safety and effective anti-tumour activity when compared to conventional treatments. However, immune checkpoint therapy can have a relatively low response rate, which has been reported to be as low as 20-30%.¹⁻³ While the precise factors resulting in these low response rates remain to be clarified, factors may include high heterogeneity of different tumour types, poor immunogenicity, and evolving capability to escape immune recognition.

### SUMMARY

Cancer is a health concern. Immune checkpoint therapy, which removes the “brake” of the immune system, has recently been used in cancer treatments. While pre-clinical studies and clinical trials have reported some success with immune checkpoint therapy, studies have reported immune checkpoint therapy a relatively low response rate.¹⁻³

One approach to addressing the low response rate of immune checkpoint therapy may be to combine immune checkpoint therapy with more traditional cancer therapies.⁴

Radiation therapy (RT) is one such traditional cancer therapy that may be a potential partner for immune checkpoint therapy. As Cuttler and Pollycove (2018) teach, “[s]trong sources of radiation became available in the 1950's. Since then, intense ionizing beams have been employed against cancer to destroy or shrink tumours. Radiation treatments for cancer have been developed based on the application of relatively high doses of radiation to local regions of the body.”

Groups have reported positive effects of localized high dose RT and immune checkpoint therapy combinations.⁴⁻⁸ However, it has also been reported that immunosuppression can result from localized high dose RT. For example, high dose RT can, in some instances, increase regulatory T-cell (T_{reg}) incidence, which can be attributed to an inherently higher radio-resistance of these cells. Induction of transforming growth factor (TGF) β secretion can also occur, which has been reported to inhibit systemic immune-activating effects. Furthermore, expression of co-inhibitory molecules such as PD-L1 has been reported to be induced in tumour cells after local high-dose RT.⁹ Therefore, high dose RT may in fact promote immunosuppression. Localized high dose RT can also result in radiation-induced side effects in normal tissues.

Contrary to high dose RT, it has been reported that low-dose whole body RT can activate the immune system by altering tumour and immune cell surface molecule expression, promoting T-cell-stimulatory capacities of dendritic cells, and/or promoting an anti-tumour macrophage phenotype.¹⁰ role for low dose RT in promoting vascular normalization within tumours has also been reported, which may enhance immunotherapeutic success by reversing the hypoxic microenvironment and enabling immune effector cell infiltration.¹¹

However, some reports using the term “low dose radiation” appear to be referring to a dose of radiation which may in fact be considered a high dose by current standards. For example, one report, which found synergistic anti-tumour effects when using 5 Gy of gamma radiation in conjunction with immune checkpoint therapy in the treatment myeloma, referred to 5 Gy of gamma radiation as “low dose radiation”.¹² Actual low dose irradiation has not been studied extensively in radiation therapy of cancers and clinical trials of low dose radiation are rare. The biological effects of low dose radiation are often extrapolated from the effects of high dose radiation from studies of atomic bomb survivors.

Despite the advances made to date in the prevention of tumour growth, there is room for improvement to address the above-mentioned problems and shortcomings of the prior art.

It is an object of the present disclosure to obviate or mitigate at least one of the above-mentioned disadvantages of the prior art.

Another object of the present disclosure is to provide a novel method of preventing tumour growth.

One broad aspect of the teachings described herein may provide for the use of a checkpoint inhibitor in combination with ultralow dose whole body irradiation in a subject for the prevention of tumour growth.

Another broad aspect of the teachings described herein may provide may provide for the use of a checkpoint inhibitor in combination with ultralow dose whole body irradiation in a subject with tumour growth for treatment of the tumour growth.

Another broad aspect of the teachings described herein may provide a method for preventing tumour growth by combining immune checkpoint therapy with ultralow dose whole body irradiation.

Thus, the present inventors have developed a treatment that comprises combining immune checkpoint therapy with ultralow dose whole body irradiation. The combination treatment provides improved anti-tumour effects by reducing tumour volume and shortening response time, as compared to immune checkpoint therapy on its own.

To the knowledge of the inventors, combining immune checkpoint therapy with ultralow dose whole body irradiation to prevent and treat tumour growth is heretofore unknown.

Other advantages of the invention will become apparent to those of skill in the art upon reviewing the present specification.

In accordance with one broad aspect of the teachings described herein, a method of preventing tumour growth in vivo in a mammalian subject, may include the steps of:
(a) administering a checkpoint inhibitor to the subject to provide a biologically active amount of the checkpoint inhibitor within the subject during a treatment period; and
(b) administering a predetermined ultralow dose of radiation to the subject via a plurality of acute, fractionated, whole body irradiation treatments delivered to the subject during the treatment period.

Another broad aspect of the teachings described herein, which may be used alone or in combination with other aspects, can include the use of a checkpoint inhibitor in combination with a predetermined ultralow dose of radiation for the prevention of tumour growth in vivo in a mammalian subject during a treatment period, wherein a biologically active amount of the checkpoint inhibitor is present within the subject during the treatment period and wherein the predetermined ultralow dose of radiation is administered to the subject via a plurality of acute, fractionated, whole body irradiation treatments delivered to the subject during the treatment period.

Other aspects of the teachings described herein, which may be used in combination with any other aspects, including the two broad aspects listed above, may include that the predetermined ultralow dose of radiation delivered during the treatment period is equal to or less than 1 Gy, and/or may be equal to or less than 100 mGy.

Consecutive ones of the plurality of acute, fractionated, whole body irradiation treatments may be separated by respective radiation inactivity periods.

Each radiation inactivity period may be between about 1 day and about 5 days, and may optionally be at least 1 day, whereby consecutive ones of the irradiation treatments are delivered every other day or may be equal to or less than 1 day, whereby consecutive ones of the irradiation treatments are delivered on consecutive days.

The plurality of acute, fractionated, whole body irradiation treatments may include 10 or fewer irradiation treatments.

The plurality of acute, fractionated, whole body irradiation treatments may include 10 irradiation treatments delivered to the subject within 36 days or within 20 days.

The radiation may be applied at a dose rate of about 0.9 Gy/hr during the irradiation treatments.

Administering the checkpoint inhibitor to the subject may include administering a plurality of doses of the checkpoint inhibitor to the subject during the treatment period.

A number of doses of the checkpoint inhibitor administered to the subject during the treatment period may be different than a number of irradiation treatments delivered to the subject during the treatment period.

At least two of the irradiation treatments may be delivered between consecutive doses of the checkpoint inhibitor.

Each irradiation treatment may be delivered to the subject on the same day as one of the doses of the checkpoint inhibitor.

Each irradiation treatment may be delivered to the subject between about 30 minutes and about 6 hours after one of the doses of checkpoint inhibitor.

Each administered dose of the checkpoint inhibitor may be between about 5 mg/kg and about 20 mg/kg, and may be about 10 mg/kg.

A cumulative dosage of the checkpoint inhibitor administered to the subject during the treatment period may be about 50 mg/kg.

The biologically active amount of the checkpoint inhibitor within the subject may be maintained by administering doses of between about 5 mg/kg and about 20 mg/kg to the subject at a frequency that is substantially equal to the half-life of the checkpoint inhibitor within the subject.

The treatment period may be between 14 and 60 days

The treatment period may be between 20 and 40 days.

The irradiation may be delivered via an irradiation source that includes cobalt-60.

The checkpoint inhibitor may be a monoclonal antibody.

The monoclonal antibody may be PD-1.

The checkpoint inhibitor may inhibit PD-L1, CTLA-4, or a combination thereof.

The checkpoint inhibitor may interact with a ligand of PD-L1, CTLA-4, or a combination thereof.

The checkpoint inhibitor may be administered subcutaneously, intraperitoneally, or intravenously.

The tumour may be associated with colon adenocarcinoma.

Each irradiation treatment may be administered to the subject while the biologically active amount of the checkpoint inhibitor is present within the subject.

### BRIEF DESCRIPTION OF THE DRAWINGS

Some embodiments of the present invention will be described with reference to the accompanying drawings, in which:

FIG. 1 illustrates a schematic diagram of an experimental design of an embodiment of the present invention, wherein a Tumour Prevention Treatment Model is used;

FIG. 2 illustrates a schematic diagram of an experimental design of an embodiment of the present invention, wherein an Established Tumour Treatment Model is used;

FIG. 3 illustrates preliminary experimental results of tumour growth under different treatments according to the experimental design illustrated in FIG. 1;

FIG. 4 illustrates preliminary experimental results of individual tumour growth under different treatments according to the experimental design illustrated in FIG. 1;

FIG. 5 illustrates the results of the tumour growth illustrated in FIG. 3 during PD-L1 and PD-L1₊ULDR treatments after the outliers have been removed;

FIG. 6 illustrates the survival rate of mice after different treatments according to the experimental design illustrated in FIG. 1;

FIG. 7 illustrates preliminary experimental results from flow cytometry analysis of CD4 and CD8 T lymphocyte populations in (A) PMBCs, (B) TILs, and (C) TILS in a treatment resistant tumour group and a treatment sensitive tumour group according to the experimental design illustrated in FIG. 1;

FIG. 8 illustrates preliminary experimental results of individual tumour growth under different treatments according to the experimental design illustrated in FIG. 2; and

FIG. 9 illustrates preliminary experimental results of individual tumour growth in (A) the combined PD-L1₊ULDR treatment and (B-E) the combined PD-L1₊ULDR treatment with immune depletion treatment according to the experimental design illustrated in FIG. 2.

### DETAILED DESCRIPTION

Various apparatuses or processes will be described below to provide an example of an embodiment of each claimed invention. No embodiment described below limits any claimed invention and any claimed invention may cover processes or apparatuses that differ from those described below. The claimed inventions are not limited to apparatuses or processes having all of the features of any one apparatus or process described below or to features common to multiple or all of the apparatuses described below. It is possible that an apparatus or process described below is not an embodiment of any claimed invention. Any invention disclosed in an apparatus or process described below that is not claimed in this document may be the subject matter of another protective instrument, for example, a continuing patent application, and the applicants, inventors, or owners do not intend to abandon, disclaim, or dedicate to the public any such invention by its disclosure in this document.

The term “checkpoint inhibitor” as used herein refers to any agent that inhibits inhibitory pathways of the immune system. Such checkpoint inhibitors may include, but are not limited to, inhibitors that bind to and inhibit immune checkpoint receptor ligands. Illustrative examples of immune checkpoint receptor ligands that may be targeted for binding and inhibiting include, but are not limited to, PD-1, PDL1, and CTLA-4.

The term “ultralow dose” as used herein refers to a dose of each radiation treatment that is between 10 mGy and 100 mGy, and the total dose received by the subject over a course of or protocol of treatment is preferably between about 100 mGy and about 1 Gy. The total dose of radiation received by the subject may be substantially the same in a given treatment protocol even if the number of radiation doses varies.

The term “subject” as used herein refers to any mammal, including but not limited to humans, primates, dogs, cats, mice, rats, farm animals, sport animals, and the like.

The term “prevention of tumour growth” as used herein is not intended as an absolute term. It is understood that the term prevention as used herein is not limited to the cure or elimination of tumour growth nor is the term limited to the achievement of certain milestones or improvement criteria in a particular subject. Instead, prevention refers to a broad range of measures, such as, but not limited to, a reduction in tumour volume, a slowing of tumour growth, and the like. All such activities are considered to be prevention whether or not any improvement is immediately observable or measurable.

The present disclosure relates, in at least one broad aspect. to the use of a checkpoint inhibitor in combination with fractioned ultralow dose whole body irradiation treatments in a subject for the prevention of tumour growth. That is, a subject that is provided with a relatively steady amount of checkpoint inhibitors during a predefined treatment period is also then subjected to a plurality of discrete, separate irradiation treatments during the treatment period, where the cumulative radiation dose that is received by the subject during the treatment period constitutes an ultralow dose of radiation. As described in detail herein, this combination of fractionated irradiation treatments to provide an ultralow doses of radiation to a subject that is also being supplied with a predetermined, relatively steady amount of a suitable checkpoint inhibitor has been found to be more effective at preventing the growth of a tumour in a mammalian subject than either of the same checkpoint inhibitor regime or fractionated radiation regime individually.

For example, in one application of the teachings described herein, a method of preventing tumour growth in vivo in a mammalian subject may include the steps of:
(a) administering a checkpoint inhibitor to the subject to provide a biologically active amount of the checkpoint inhibitor within the subject during a treatment period; and
(b) administering a predetermined ultralow dose of radiation to the subject via a plurality of acute, fractionated, whole body irradiation treatments delivered to the subject during the treatment period and while the biologically active amount of the checkpoint inhibitor is present within the subject.

Another application of the teachings described herein may include the use of a checkpoint inhibitor in combination with a predetermined ultralow dose of radiation for the prevention of tumour growth in vivo in a mammalian subject during a treatment period, wherein a biologically active amount of the checkpoint inhibitor is present within the subject during the treatment period and wherein the predetermined ultralow dose of radiation is administered to the subject via a plurality of acute, fractionated, whole body irradiation treatments delivered to the subject during the treatment period.

While some examples of particular combinations and dosages of checkpoint inhibitors and irradiation treatments have been tested and are described in detail herein, various other analogous combinations of different specific dosages of checkpoint inhibitors and corresponding administration schedules may be used with a common irradiation therapy protocol, and/or various combinations different irradiation treatment schedules and individual treatment radiation dosages may be utilized with a generally constant checkpoint inhibitor regime. That is, different combinations of checkpoint inhibitor and irradiation regimes may provide varying degrees of tumour growth prevention in a given subject.

In certain embodiments, the checkpoint inhibitor used is a monoclonal antibody, but alternatively may include chemical small molecule inhibitor.

In certain embodiments, the checkpoint inhibitor inhibits PD-L1, CTLA-4, or a combination thereof. Preferably, the checkpoint inhibitor interacts with a ligand of PD-L1, CTLA-4, or a combination thereof. Optionally, the checkpoint inhibitor may be PD-1.

The checkpoint inhibitor may be administered using any suitable technique(s), and may optionally be administered as a single treatment or as a plurality of treatments that are spaced apart from each other. For example, the checkpoint inhibitor may be applied during one or more administration phases or periods that are separated from each other by periods of inactivity/recovery. Such inactivity periods may be of any suitable length, and may be hours, days, weeks, or more. For example, if the checkpoint inhibitor is administered twice a week, the inactivity period between administration periods may be 2-4 days each.

In certain embodiments of the invention, the checkpoint inhibitor is administered through a plurality of treatments in respective administration periods. In certain embodiments, the checkpoint inhibitor may be administered once month, once every two weeks, once a week, twice a week, three times a week, four times a week, five times a week, six times a week, daily, and the like. The inactivity period between checkpoint inhibitor treatments may be selected based on a variety of factors, including, for example the half life of the antibody administered, which may vary based on the inhibitor selected and the type of subject being treated. In certain embodiments, the checkpoint inhibitor is administered for one week, two weeks, three weeks, four weeks, five weeks, six weeks, two months, and the like. Preferably, the checkpoint inhibitor is administered for a treatment period of at least five weeks, but in some instances may be administer for a shorter time period. Preferably, as described herein, the inactivity period between checkpoint inhibitor treatments may be selected so that the amount of the checkpoint inhibitor within the subject remains generally constant during the course of a given treatment period.

The inactivity periods between subsequent administration periods for the checkpoint inhibitor may be generally the same as each other (i.e., the administrations of the checkpoint inhibitor may be generally equally spaced from each other), or may be of different lengths if the checkpoint inhibitor is administered with non-constant timing/frequency. The inactivity between administration periods may be hours, or may be one day, two days, three days, four days, five days, six days, seven days, or more, as desired for a given embodiment of the teachings described herein.

Optionally, the checkpoint inhibitor is administered in doses of between about 5 mg/kg and about 20 mg/kg, or any combination of doses thereof. Preferably, the checkpoint inhibitor may be administered at a dosage of about 5 mg/kg and more preferably at a dosage of about 10 mg/kg.

The dosage of the checkpoint inhibitor may be the same for each administration period. For example, a constant dosage of checkpoint inhibitor may be provided over the course of a given treatment protocol. Alternatively, the dosage of checkpoint inhibitor administered may be different at different stages of the treatment protocol.

Optionally, in addition to selecting a given dosage per administration period, a cumulative dose of the checkpoint inhibitor that is to be administer to a given subject over a treatment course/protocol can be about 50 mg/kg, or about 200 mg/kg (or possibly other amounts in other circumstances). Optionally, the checkpoint inhibitor may be administered at cumulative dosage of about 50 mg/kg.

Preferably, the checkpoint inhibitor is administered in such a manner that there remains at least a minimum effective amount of the checkpoint inhibitor within the subject during relevant portions of the treatment period, such as when the subject is subjected to the irradiation treatments as part of the cumulative radiation therapy as described herein. In some subjects, the target biologically active and/or effective amount of the checkpoint inhibitor that is desired to be present in the subject during radiation therapy may be provided by administering the checkpoint inhibitor in accordance with a predetermined dosage regime. For example, the biologically effective amount of the checkpoint inhibitor may be established by administering dosages of at least 5 mg/kg, at least 10 mg/kg, at least 15 mg/kg, at least 20 mg/kg, or more on a frequency that may generally coincide with the expected half-life of the particular checkpoint inhibitor that is being used. This may help provide a generally steady amount of checkpoint inhibitor during the treatment period.

It may also be desirable in some circumstances to administer the checkpoint inhibitor and an irradiation treatment on the same day, for example if the subject would have to visit a hospital or other facility to receive the treatments. To help provide the desired amount of checkpoint inhibitor within the subject when the irradiation treatment is conducted, it may be desirable to wait a buffer time period between the administration of the checkpoint inhibitor and the administration of the irradiation treatment. Optionally, the buffer time may be between about 30 minutes and about 6 hours or more.

Optionally, the checkpoint inhibitor may be administered subcutaneously, intraperitoneally, intravenously, any combination thereof, or using another suitable technique. The administration technique may be determined based on the subject being treated, the dosage of checkpoint therapy to be administered, or other factors.

In combination with the administration of a suitable checkpoint therapy, the subject being treated may receive radiation therapy that can include administering a predetermined ultralow dose of radiation to the subject via a plurality of acute, fractionated, whole body irradiation treatments delivered to the subject during the treatment period. As discussed herein, the present inventors have discovered that the combination of a radiation treatment of this type used in combination with a suitable checkpoint inhibitor (i.e., such that the radiation is administered to a subject that has already received a predetermined, target dose of the checkpoint inhibitor) can help prevent tumour growth more effectively than the use of either the checkpoint inhibitor or radiation therapy described herein in isolation.

Preferably, the radiation therapy delivery schedule is set so that the subject will receive a pre-determined, cumulative dose of radiation over the course of the treatment period (e.g. the period that includes the first and the last scheduled radiation treatments in a given course or protocol for treatment) via two or more separate irradiation treatments. The cumulative dose received in accordance with the teachings described herein is preferably an ultralow dose of radiation as defined below.

More preferably, the radiation therapy may be administered in one or more fractionated irradiation treatments, each delivered during a corresponding radiation administration period over the course of a treatment protocol, separated by suitable radiation recovery or radiation inactivity periods. The cumulative dose of radiation may be distributed evenly, or unevenly between the irradiation treatments, provided that the sum of the radiation dosages from the set of fractionated treatments equals the desired cumulative dosage. The number of radiation administration periods and intervening radiation inactivity periods may be varied in different implementations of the teachings described herein.

The radiation administration periods may be performed at the same frequency and duration as the checkpoint therapy, or at a different frequency and/or for a different duration. Preferably, the administration of the checkpoint inhibitor is conducted in a manner so that a relatively constant amount of the checkpoint inhibitor is present within the subject during each irradiation treatment. That is, the irradiation treatments are each preferably conducted while the subject has approximately the same generally biologically active amount of the checkpoint inhibitor in its system. In circumstances where the actual active concentration of the checkpoint inhibitor in the subject is difficult to determine, a generally constant level of checkpoint inhibitor within the subject may be inferred from a generally constant dosing regime, including those described herein. As noted above, taking steps to help ensure that a generally constant amount of checkpoint inhibitor is administered to the subject may help facilitate generally constant conditions during each irradiation treatment during the treatment period.

Similarly, the radiation inactivity periods may be the same as the checkpoint therapy inactivity periods or may be different. Preferably, the radiation and checkpoint therapy are administered to the subject on substantially the same frequency. That is, the checkpoint therapy administration period may at least partially, substantially, and/or completely overlap with the radiation administration period. For example, both the checkpoint therapy and a dose of radiation may be administered to a subject on the same day. Alternatively, the checkpoint therapy and radiation may be administered on different schedules and during different periods. For example, the administration period for the checkpoint therapy may overlap with a radiation inactivity period, or vice versa.

Optionally, the radiation may be administered through a plurality of treatments in respective radiation periods. In one of the embodiments described herein, the radiation was administered twice a week over a treatment period that spanned about 5 weeks. In another embodiment, the same number of irradiation treatments, totalling the same cumulative dosage of radiation may be delivered in a shorter treatment period. For example, irradiation treatments may be administered every other day for a period of about two weeks.

In some circumstances, the total number of irradiation treatments can vary based on a desired treatment schedule and in view of the desired cumulative dosage. Optionally, in other embodiments, the radiation may be administered with a different frequency.

In certain embodiments, the course of treatment (i.e. the treatment period) during which radiation may be administered may be any suitable time, and may be more than about 7 days, 10 days, 11 days, 12 days, 13 days, 14 days, 15 days, 16 days, 17, days, 18 days, 19 days, 20 days, or more and may be less than about 90 days, 80 days, 70 days, 60 days, 50 days, 40 days, 35 days, 30 days, 25 days, 20 days, 18 days, 16, days, 14 days, or fewer. In an embodiment tested and described herein, the course of treatment of during which radiation was administered was about five weeks.

For example, the irradiation treatments may include 10 irradiation treatments delivered to the subject in a treatment period of 36 days, or 10 irradiation treatments delivered to the subject in a treatment period of 20 days, or 14 days or fewer, but the subject may receive the same cumulative radiation dose in the treatment periods.

Optionally, the checkpoint inhibitor may be administered prior to the administration of the radiation. In the embodiments described herein and as tested by the inventors, a checkpoint inhibitor was injected into subjects on the same day and at least 30 minutes prior to the delivery of the radiation dose. In other embodiments, the interval of time between the administration of the checkpoint inhibitor and the radiation may be different.

Optionally, the checkpoint inhibitor may be administered at substantially the same time as the radiation (i.e. within less than 15 minutes of the radiation dosage), and optionally may be administered simultaneously with the radiation.

Optionally, the checkpoint inhibitor may be administered after the administration of the radiation, for example about 30 minutes after the administration of the radiation.

Preferably, the radiation administered using the protocols described herein can be administered in ultralow dosage ranges, which may be below the range of radiation dosage that is associated with conventional cancer treatment regimes. For example, in the embodiment describe herein, the radiation may be administered at a dose rate of about 0.9 Gy/hour, or other rates may be suitable under some circumstances.

Optionally, the cumulative dose of irradiation that is administered may be about 100 mGy to about 1000 mGy. Preferably, the radiation may be administered at a cumulative dosage of about 100 mGy. Optionally, the radiation may be administered acutely.

The radiation source used in the treatments described herein may be any suitable source, and may include cobalt-60.

In certain embodiments of the invention, the tumour that is being treated using the combination of ultralow dose radiation and checkpoint therapy may be associated with colon adenocarcinoma.

### Experimental Examples

In accordance with some of the broad aspects of the teaching described herein, the following examples are provided to illustrate exemplary embodiments of the present invention but should not be used to limit or construe other embodiments of the teachings described herein.

Reagents

Dulbecco's modified Eagle's medium (DMEM) and Roswell Park Memorial Institute medium (RPMI) were purchased from HyClone (Fisher Scientific, Toronto, ON, Canada). Ethanol and methanol were purchased from Commercial Alcohols, Inc. (Brampton, ON, Canada).

### Animals

Adult female BALB/c mice, aged 2 months, were purchased from Jackson Laboratory, Bar Harbor, Me. Mice were randomly assigned into groups of six and allowed to acclimatize to the facility for a period of two weeks. Animals were housed in a specific pathogen-free environment in the Biological Research Facility (BRF) at Canadian Nuclear Laboratories, in Chalk River, Ontario, Canada. Mice were maintained in filter-top cages on ventilated cage racks equipped with an automatic watering system. Animal health was assessed by animal care staff on a daily basis and mice were fed autoclaved Rodent Chow #5075 (Charles River, Canada) ad libitum. The BRF was equipped with automatic computer-controlled temperature (23° C.), air ventilation, and a 12 hr light/dark cycle. Routine health monitoring tests were performed to screen for infections and the presence of pathogens and mice were determined to be pathogen and infection free before participating in the study. During euthanization, visual examination and complete blood profile tests were performed to determine which mice, if any, were to be excluded from the results due to poor health conditions (unrelated to treatment). All protocols were performed in accordance with the guidelines of the Canadian Council on Animal Care and with the approval of the local Animal Care Committee.

### Cell Lines

CT26, a colon adenocarcinoma cell line, was purchased from American Type Culture Collection (ATCC). It was tested for murine and human pathogens by Charles River Canada and mycoplasma with MycoAlert Mycoplasma Detection Kit.

### Tumour Growth and Treatments

Mice were anesthetized with isoflurane (Abbott Laboratories, Chicago, Ill.) and then injected s.c. with CT-26 tumour cells (1×10⁵ in 100 ul PBS) in the right flank (Day 0). Animals were subsequently monitored for deteriorating health for a minimum of one hour post injection.

In the illustrated example, two treatment models were studied—a Tumour Prevention Treatment Model and an Established Tumour Treatment Model.

In the Tumour Prevention Treatment Model, to examine the effects of ULDR on the anti-tumour effects of anti-PD-L1 treatment, both PD-L1 (Bioxcell, 5 mg/kg, i.p. in 100 μl PBS) and gamma beam whole body radiation treatments (GC60-1000, Cobalt-60, 0.94 Gy/hr, Canadian Nuclear Laboratories, Chalk River, ON, Canada) began one day after inoculation with CT-26 tumour cells.

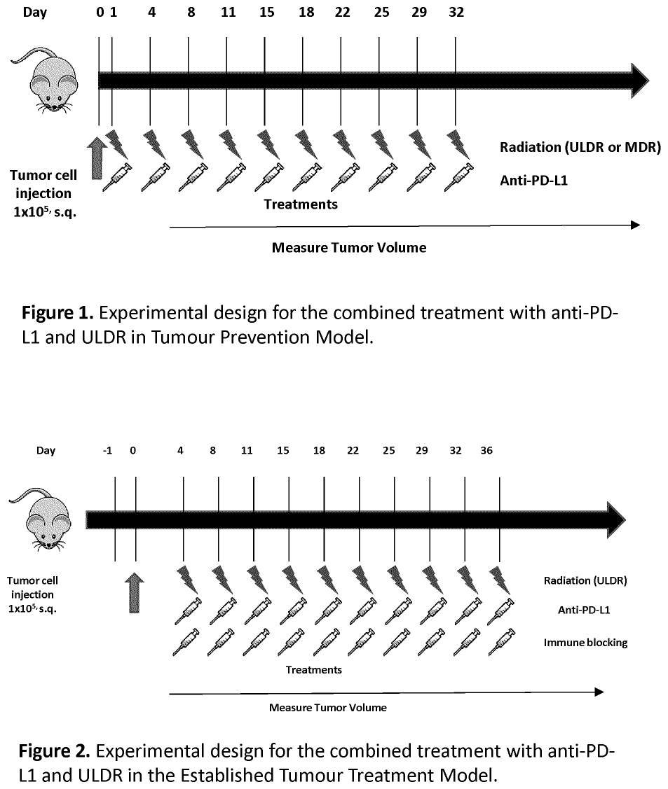

FIG. 1 illustrates a schematic diagram of an experimental design of an embodiment of the present invention using the Tumour Prevention Treatment Model. Mice in the Tumour Prevention Treatment Model were divided into the following treatment groups:

1. a control antibody and sham radiation treatment (Control);

2. ultralow dose radiation at 10 mGy with control antibody (ULDR);

3. medium dose radiation at 100 mGy with control antibody (MDR);

4. anti-PDL1 antibody at 5 mg/kg and sham radiation (PD-L1);

5. ULDR with anti-PD-L1 antibody (PD-L1₊ULDR); and

6. MDR with anti-PD-L1 antibody (PD-L1₊MDR).

Mice were irradiated twice a week for five weeks, for a total of 10 exposures. Prior to irradiation, treatment groups, including the radiation treatment sham, were placed in sterile cages and moved to the “gamma hall” within the BRF animal facility to receive radiation. After irradiation, mice were returned to their original cages and maintained in the animal housing facility. Tumour volumes were measured three times a week using digital calipers, and tumour volume was calculated using the following equation volume=a*b*b/2, where a represents the largest diameter and b represents the diameter perpendicular to a.

For mice in the Established Tumour Treatment Model, both PD-L1 (Bioxcell, 5 mg/kg, i.p. in 100 μl PBS) and gamma beam whole body radiation treatments (GC60-1000, Cobalt-60, 0.94 Gy/hr, Canadian Nuclear Laboratories, Chalk River, ON, Canada) began four days after inoculation with CT-26 tumour cells. By delaying treatment for four days, tumours in the Established Tumour Treatment Model group were allowed to reach a volume of 50-100 mm³ before treatment commenced. In other words, mice in the Established Tumour Treatment Model received immune checkpoint therapy treatment after the tumour was established. In addition, “immune depletion treatments” were performed in the Established Tumour Treatment Model, wherein specific immune cells were depleted. Treatment included antibodies against CD4 (BioXcell, clone GK1.5)⁵, CD8 (BioXcell, clone 53-6.7)^{5,6} or both⁵, injected intraperitoneally (i.p.) at a concentration of 3 mg/kg, anti-Asialo GM1 (Wako)^{6,13}, injected i.p. at 10 μl/mouse, to reduce T-cell and natural killer (NK) cell populations respectively. Control IgG2B antibodies were administered to the control group via i.p. injection at a concentration of 3 mg/kg. Immune depletion treatments coincided with PD-L1 treatments.

FIG. 2 illustrates a schematic diagram of an experimental design of an embodiment of the present invention using the established Tumour Treatment Model. In the Established Tumour Treatment Model, treatment continued twice per week for 6 weeks.

Each group consisted of 12 mice.

### Blood & Plasma Collection

Mice were anesthetized with isoflurane in a rodent anesthesia induction chamber, and whole blood was collected via cardiac puncture and stored in pre-coated EDTA blood collection tubes. Whole blood was centrifuged at 1500×g for 15 min and the blood plasma was subsequently collected and frozen at −80° C.

### PBMC Purification

After collection, the blood pellet was diluted with 1 ml PBS containing 10 mM EDTA and mixed via inversion. SepMate™-15 tubes (STEMCELL Technologies Canada Inc., Vancouver, BC) were prepared by adding 5 mL of Lymphoprep density gradient medium (STEMCELL Technologies Canada Inc., Vancouver, BC) to each tube and centrifuging at 1000 g for 1 min. Diluted blood was pipetted down the side of each tube, making sure tubes remained vertical, to prevent unwanted mixing. Loaded SepMate™ tubes were centrifuged at 1200 g for 10 min at room temperature and the top layer, containing enriched PBMCs, was decanted into 15 mL centrifuge tubes. PBS containing 2% FBS and 10 mM EDTA was added to the MNCs and centrifuged at 2000 g for 10 min at room temperature. The resulting supernatant was discarded, and the cell pellet was suspended via repeated pipetting in 2 mL of RPMI 1640 (Lonza, Slough, UK) containing 5% DMSO and 20% FBS and was subsequently frozen in two aliquots at −80° C.

### Tumour Infiltration Lymphocytes Purification

After euthanization, tumours were excised from the right flank and dissociated according to the GentleMACS dissociation protocol. Briefly, tumours were dissected into fragments, 2-3 mm in length, and transferred to C-tubes (Miltenyi Biotech) containing 10 mL of RPMI 1640 (Lonza, Slough, UK) and enzymes D, R, A (Miltenyi Biotec) to form a digest mix. The mix was mechanically disaggregated in the GentleMACS Dissociator for two 36-second steps (programs m_tumour_02 and m_tumour_03) with a 60-min incubation at 37° C. and 37 RPM in between. The disaggregated tumour mixture was transferred to a 50 mL centrifuge tube, passing through a 40 μm cell strainer, and the remaining liquid containing the tumour infiltration lymphocytes (TIL) was subsequently centrifuged at 2000 g for 10 min. The resulting supernatant was discarded and the cell pellet re-suspended via repeated pipetting in 2 mL of RPMI 1640, containing 5% DMSO and 20% FBS, and then frozen in two aliquots at −80° C.

### Flow Cytometry

PBMCs and TILs were stained with anti-CD3 (BD Horizon BV510 Hamster Anti-Mouse CD3e), anti-CD4 (FITC Rat Anti-Mouse CD4), anti-CD8 (BD Pharmingen APC-H7 Rat anti-mouse CD8a), and anti-CD25 (APC rat anti-mouse CD25 Clone PC61 (RUO)). Cells were first stained for indicated cell surface markers, then fixed in 200 μL of 1% Paraformaldehyde (PFA). Flow cytometry was performed using a FACSCelesta flow cytometer (Becton Dickinson, USA) and analyzed using FlowJo software V.10 (Tri-Star, USA).

### Statistical Analysis

All data sets were expressed as mean±standard error of the mean (SEM). All experiments were performed independently at least twice and biological replicates were used for each data set. GraphPad Prism v. 6.07 for windows was used for data analysis. Statistical significance was considered at p<0.05.

### Results

### Combination of Anti-PD-L1 Treatment and ULDR in the Tumour Prevention Treatment Model

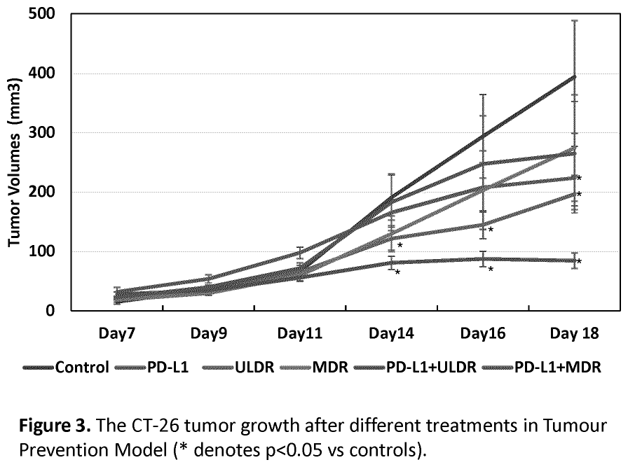

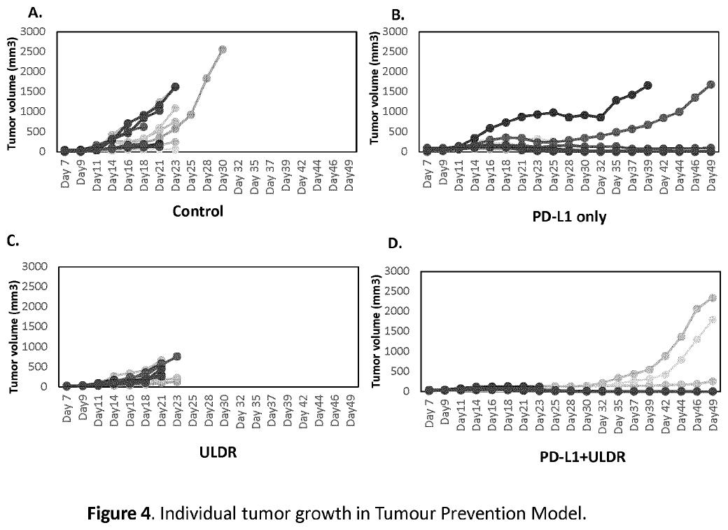

FIG. 3 illustrates preliminary experimental results of the illustrated embodiment of the present disclosure in which the effects of anti-PD-L1 treatment in combination with ultralow dose radiation on tumour growth in vivo was examined in the Tumour Prevention Treatment Model. As above, CT-26 cells (1×10⁵) were inoculated s.q. into the right flanks of mice, and treatments were started one day after the inoculation. The anti-tumour effects of the combination therapy were determined using the tumour growth delay.

As illustrated in FIG. 3, compared to the control group, tumour growth suppression was significant in the ULDR₊PD-L1 combined treatment group on Days 14, 16, and 18 (P<0.05). Likewise, ULDR only treatment reduced tumour growth on Days 14, 16, and 18, as compared to the control group (P<0.05). However, tumour growth suppression was not significant in the anti-PD-L1 alone group, as compared to the control group, until Day 18. MDR had no significant effect on tumour growth (P>0.05).

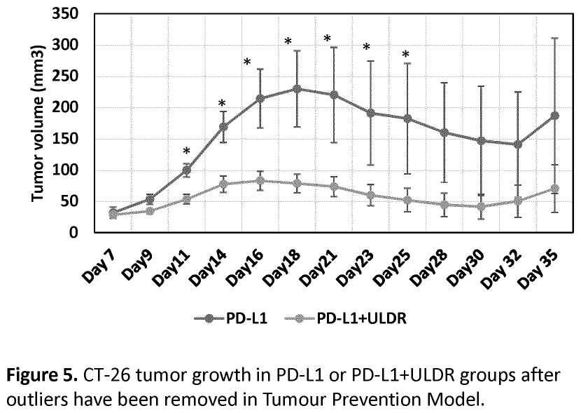

In mice treated with PD-L1₊ULDR, average tumour volume on day 18, when all mice were still alive, was 84.26 mm³ compared with 393.45 mm³ in the control group, 223.29 mm³ in the PD-L1 group, and 195.99 mm³ in the ULDR group, 273.91 mm³ in the MDR group, and 264.4 mm³ in the PD-L1₊MDR group. From this, tumour volume in the ULDR₊PD-L1 combined treatment was significantly less than that in the ULDR only treatment and in the anti-PD-L1 only treatment (p<0.05). Therefore, the combination treatment of ULDR with anti-PD-L1 suppressed tumour growth as compared to immune checkpoint therapy on its own and as compared to ULDR treatment on its own. Individual tumour growth is shown in FIG. 4.

Starting from Day 18, some mice, either with bigger tumours or at risk for ulceration, were euthanized based on the guidelines pre-determined by the Animal Protocols and SOPs by the local animal care committee. One mouse from the PD-L1 treatment group and two mice from the PD-L1₊ULDR treatment group were sacrificed due to the risk of ulceration.

FIG. 5 illustrates preliminary experimental results of CT-26 tumour growth during the PD-L1 and PD-L1₊ULDR treatments illustrated in FIG. 3 after the outliers were removed. Both treatments slowed tumour growth, and tumour shrinkage (decrease of tumour volume) was observed at Day 21 for PD-L1 treatment group and at Day 18 for the PD-L1₊ULDR treatment group. Average tumour volume, from Day 11 to 25, was significantly less in mice receiving PD-L1₊ULDR when compared to mice receiving PD-L1 alone (p<0.05).

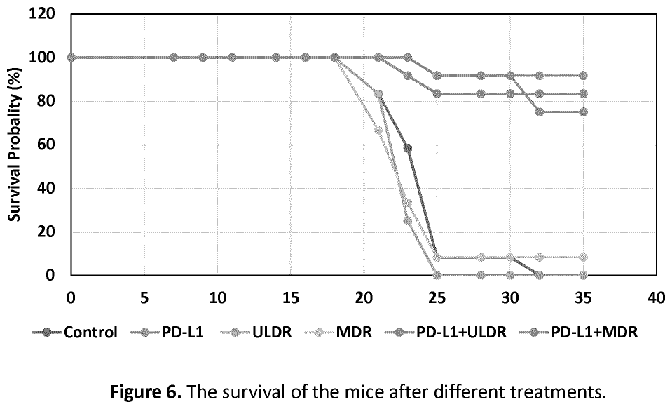

Following the final treatment (Day 32), tumour volume was measured to compare the long-term effects of treatment. As illustrated in FIG. 6, there was no significant difference in survival between mice treated with PD-L1 alone and those treated with PD-L1₊ULDR (p>0.05).

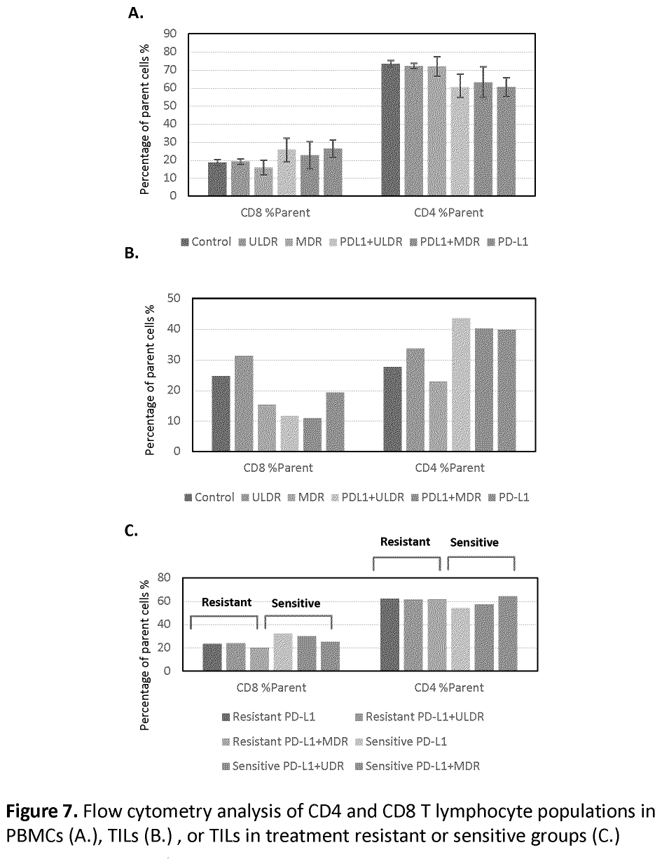

FIG. 7 illustrates preliminary experimental results of immune phenotype of blood PBMCs across treatment groups using surface expression on CD4 and CD8 T lymphocytes as measured with flow cytometry. The percentage of CD8 T cells found in parental cells (CD3 positive T cells) was significantly greater in mice treated with PD-L1 (p<0.05), but did not differ significantly in mice treated with ULDR (p>0.05), as compared to controls.

As shown in FIG. 7C, immune phenotype of blood tumour infiltration lymphocytes (TIL) was also compared across treatment groups using surface expression on CD4 and CD8 T lymphocytes measured with flow cytometry. The PD-L1, PD-L1₊ULDR, and PD-L1₊MDR treatment groups were separated into “treatment sensitive tumours” (i.e., tumour volume after treatment was similar or smaller than that before treatment) or “treatment resistant tumours” (i.e., tumour volume after treatment was larger than that before treatment). Treatment sensitive tumours were found to have more CD8 TILs than those of the treatment resistance tumours. Percentages of CD8 TILs were significantly greater in mice with treatment sensitive tumours than in mice with treatment resistant tumours (p<0.05).

### Combination of Anti-PD-L1 Treatment and ULDR in the Established Tumour Treatment Model

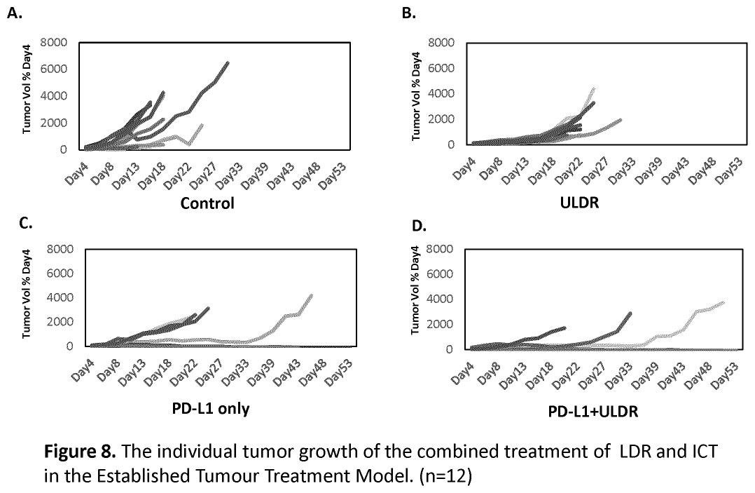

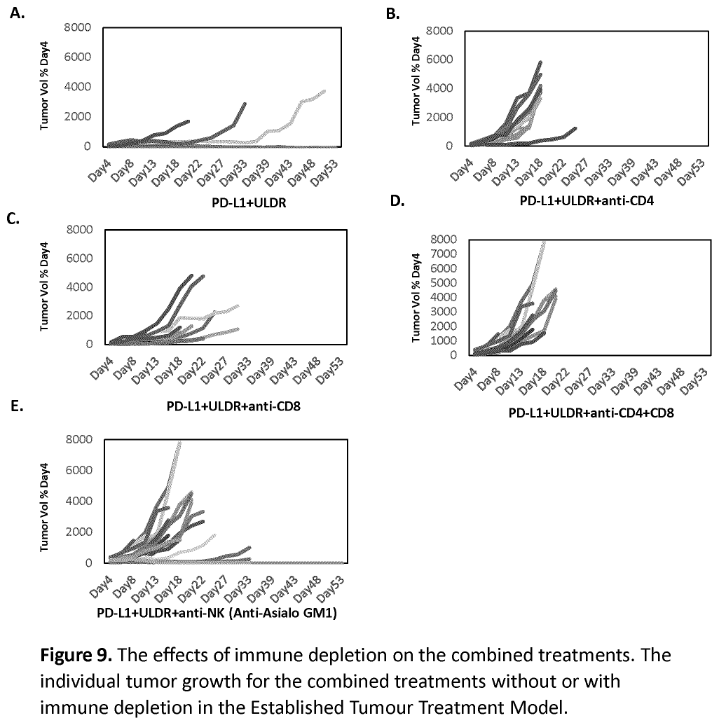

FIG. 8 illustrates preliminary experimental results of the effect of anti-PD-L1 treatment in combination with ultralow dose radiation on tumour growth in the Established Tumour Treatment Model. Tumour growth was significantly delayed in mice treated with ULDR, when compared to the control group (p<0.05). Similarly, tumour growth was significantly lower in mice treated with PD-L1 alone or PD-L1₊ULDR (p<0.05), compared to other treatments, with the lowest rate of growth measured in mice treated with PD-L1₊ULDR.

### Immune Depletion in the Established Tumour Treatment Model

To better understand the effectiveness of anti-PD-L1 treatment in combination with ultralow dose radiation, levels of three immune cells (either CD4, CD8 or NK cells) were significantly reduced prior to and throughout treatment. Temporary immunosuppression did not cause mice to become immunocompromised and mice did not required special treatment beyond the standard BRF animal housing procedures.

FIG. 8A illustrates individual tumour growth when mice received anti-PD-L1 treatment in combination with ultralow dose radiation and FIGS. 8B-E illustrate individual tumour growth when mice received anti-PD-L1 treatment in combination with ultralow dose radiation when also subjected to immune depletion. Among the groups treated with PD-L1₊ULDR, those with reduced CD4 and CD8 T cell lines had the lowest levels of tumour regression, followed by those with reduced CD4 or CD8 T cells, and then those with reduced NK cells only.

### Discussion

Cancer cells normally activate the immune checkpoint pathway to block anti-tumour immunity. Immune checkpoint therapy removes the “brake” of the immune system and enhances the body's immune function against tumours. In the illustrated embodiment, LDR improved the anti-tumour effects of PD-L1 treatment in both the Preventative Treatment Model and the Established Tumour Treatment Model. Furthermore, mice experienced smaller tumour volumes and greater responsiveness to PD-L1 therapy.

The results discussed herein suggest that ULDR radiation may be a viable alternative to high dose RT, may be an effective adjunct to immune checkpoint therapy, and may enhance the anti-cancer effects of immune checkpoint therapy.

The effectiveness of immune checkpoint therapy as a cancer therapy may be reliant on the activation and mobilization of the immune system to inhibit the tumour growth. In the illustrated embodiment, mice with depleted levels of CD4, CD8 T cells and NK cells experienced significantly delayed treatment responsiveness, lower levels of tumour reduction, and greater tumour volumes.

While this invention has been described with reference to illustrative embodiments and examples, the description is not intended to be construed in a limiting sense. Thus, various modifications of the illustrative embodiments, as well as other embodiments of the invention, will be apparent to persons skilled in the art upon reference to this description. It is therefore contemplated that the appended claims will cover any such modifications or embodiments.

All publications, patents and patent applications referred to herein are incorporated by reference in their entirety to the same extent as if each individual publication, patent, or patent application was specifically and individually indicated to be incorporated by reference in its entirety.

### LIST OF REFERENCES CITED

1. Soukup, K. & Wang, X. Radiation meets immunotherapy—a perfect match in the era of combination therapy? Int J Radiat Biol 91, 299-305 (2015).
2. Brahmer, J. R. & Pardoll, D. M. Immune checkpoint inhibitors: making immunotherapy a reality for the treatment of lung cancer. Cancer Immunol Res 1, 85-91 (2013).
3. Kalbasi, A., June, C. H., Haas, N. & Vapiwala, N. Radiation and immunotherapy: a synergistic combination. J Clin Invest 123, 2756-63 (2013).
4. Takahashi, Y. et al. Radiation enhanced the local and distant anti-tumor efficacy in dual immune checkpoint blockade therapy in osteosarcoma. PLoS One 12, e0189697 (2017).
5. Oweida, A. et al. Ionizing radiation sensitizes tumors to PD-L1 immune checkpoint blockade in orthotopic murine head and neck squamous cell carcinoma. Oncoimmunology 6, e1356153 (2017).
6. Kim, K. J. et al. Radiation improves antitumor effect of immune checkpoint inhibitor in murine hepatocellular carcinoma model. Oncotarget 8, 41242-41255 (2017).
7. Twyman-Saint Victor, C. et al. Radiation and dual checkpoint blockade activate non-redundant immune mechanisms in cancer. Nature 520, 373-7 (2015).
8. Deng, L. et al. Irradiation and anti-PD-L1 treatment synergistically promote antitumor immunity in mice. J Clin Invest 124, 687-95 (2014).
9. Klug, F. et al. Low-dose irradiation programs macrophage differentiation to an iNOS(₊)/M1 phenotype that orchestrates effective T cell immunotherapy. Cancer Cell 24, 589-602 (2013).
10. Farooque, A. et al. Low-dose radiation therapy of cancer: role of immune enhancement. Expert Rev Anticancer Ther 11, 791-802 (2011).
11. Cui, J. et al. Hormetic Response to Low-Dose Radiation: Focus on the Immune System and Its Clinical Implications. Int J Mol Sci 18(2017).
12. Jing, W. et al. Combined immune checkpoint protein blockade and low dose whole body irradiation as immunotherapy for myeloma. J Immunother Cancer 3, 2 (2015).
13. Monnier, J. & Zabel, B. A. Anti-asialo GM1 NK cell depleting antibody does not alter the development of bleomycin induced pulmonary fibrosis. PLoS One 9, e99350 (2014).

What is claimed is:

1. A method of preventing tumour growth in vivo in a mammalian subject, the method comprising:
(a) administering a checkpoint inhibitor to the subject to provide a biologically active amount of the checkpoint inhibitor within the subject during a treatment period; and
(b) administering a predetermined ultralow dose of radiation to the subject via a plurality of acute, fractionated, whole body irradiation treatments delivered to the subject during the treatment period.

2. The method of claim 1, wherein the predetermined ultralow dose of radiation delivered during the treatment period is equal to or less than 1 Gy.

3. The method of claim 1 or 2, wherein the predetermined ultralow dose of radiation delivered during the treatment period is equal to or less than 100 mGy.

4. The method of any one of claims 1 to 3, wherein consecutive ones of the plurality of acute, fractionated, whole body irradiation treatments are separated by respective radiation inactivity periods.

5. The method of any one of claims 1 to 4, wherein each radiation inactivity period is between about 1 day and about 5 days.

6. The method of any one of claims 1 to 5, wherein each radiation inactivity period is at least 1 day, whereby consecutive ones of the irradiation treatments are delivered every other day.

7. The method of any one of claims 1 to 4, wherein each radiation inactivity period is equal to or less than 1 day, whereby consecutive ones of the irradiation treatments are delivered on consecutive days.

8. The method of any one of claims 1 to 7, wherein the plurality of acute, fractionated, whole body irradiation treatments comprises 10 or fewer irradiation treatments.

9. The method of any one of claims 1 to 8, wherein the plurality of acute, fractionated, whole body irradiation treatments comprises 10 irradiation treatments delivered to the subject within 36 days.

10. The method of any one of claims 1 to 8, wherein the plurality of acute, fractionated, whole body irradiation treatments comprises 10 irradiation treatments delivered to the subject within 20 days.

11. The method of any one of claims 1 to 10, wherein the radiation is applied at a dose rate of about 0.9 Gy/hr during the irradiation treatments.

12. The method of any one of claims 1 to 11, wherein administering the checkpoint inhibitor to the subject comprises administering a plurality of doses of the checkpoint inhibitor to the subject during the treatment period.

13. The method of claim 12, wherein a number of doses of the checkpoint inhibitor administered to the subject during the treatment period is different than a number of irradiation treatments delivered to the subject during the treatment period.

14. The method of claim 12 or 13, wherein at least two of the irradiation treatments are delivered between consecutive doses of the checkpoint inhibitor

15. The method of claim 12 or 13, wherein each irradiation treatment is delivered to the subject on the same day as one of the doses of the checkpoint inhibitor.

16. The method of claim 15, wherein each irradiation treatment is delivered to the subject between about 30 minutes and about 6 hours after one of the doses of checkpoint inhibitor.

17. The method of any one of claims 12 to 16, wherein each administered dose of the checkpoint inhibitor is between about 5 mg/kg and about 20 mg/kg.

18. The method or use according to claim 17, wherein each administered dose of the checkpoint inhibitor is about 5 mg/kg.

19. The method of any one of claims 1 to 18, wherein a cumulative dosage of the checkpoint inhibitor administered to the subject during the treatment period is about 50 mg/kg.

20. The method of any one of claims 1 to 19, wherein the biologically active amount of the checkpoint inhibitor within the subject is maintained by administering doses of between about 5 mg/kg and about 20 mg/kg to the subject at a frequency that is substantially equal to the half-life of the checkpoint inhibitor within the subject.

21. The method of any one of claims 1 to 20, wherein the treatment period is between 14 and 60 days.

22. The method of any one of claims 1 to 21, wherein the treatment period is between 20 and 40 days.

23. The method of any one of claims 1 to 22, wherein the irradiation is delivered via an irradiation source that comprises cobalt-60.

24. The method of any one of claims 1 to 23, wherein the checkpoint inhibitor is a monoclonal antibody.

25. The method of claim 24, wherein the monoclonal antibody is PD-1.

26. The method of any one of claims 1 to 25, wherein the checkpoint inhibitor inhibits PD-L1, CTLA-4, or a combination thereof.

27. The method of any one of claims 1 to 26, wherein the checkpoint inhibitor interacts with a ligand of PD-L1, CTLA-4, or a combination thereof.

28. The method of any one of claims 1 to 27, wherein the checkpoint inhibitor is administered subcutaneously, intraperitoneally, or intravenously.

29. The method of any one of claims 1 to 28, wherein the tumour is a tumour associated with colon adenocarcinoma.

30. The method of any one of claims 1 to 29, wherein each irradiation treatment is administered to the subject while the biologically active amount of the checkpoint inhibitor is present within the subject.

31. Use of a checkpoint inhibitor in combination with a predetermined ultralow dose of radiation for the prevention of tumour growth in vivo in a mammalian subject during a treatment period, wherein a biologically active amount of the checkpoint inhibitor is present within the subject during the treatment period and wherein the predetermined ultralow dose of radiation is administered to the subject via a plurality of acute, fractionated, whole body irradiation treatments delivered to the subject during the treatment period.

32. The use of claim 31, wherein the predetermined ultralow dose of radiation delivered during the treatment period is equal to or less than 1 Gy.

33. The use of claim 31 or 32, wherein the predetermined ultralow dose of radiation delivered during the treatment period is equal to or less than 100 mGy.

34. The use of any one of claims 31 to 33, wherein consecutive ones of the plurality of acute, fractionated, whole body irradiation treatments are separated by respective radiation inactivity periods.

35. The use of any one of claims 31 to 34, wherein each radiation inactivity period is between about 1 day and about 5 days.

36. The use of any one of claims 31 to 35, wherein each radiation inactivity period is at least 1 day, whereby consecutive ones of the irradiation treatments are delivered every other day.

37. The use of any one of claims 31 to 34, wherein each radiation inactivity period is equal to or less than 1 day, whereby consecutive ones of the irradiation treatments are delivered on consecutive days.

38. The use of any one of claims 31 to 37, wherein the plurality of acute, fractionated, whole body irradiation treatments comprises 10 or fewer irradiation treatments.

39. The use of any one of claims 31 to 38, wherein the plurality of acute, fractionated, whole body irradiation treatments comprises 10 irradiation treatments delivered to the subject within 36 days.

40. The use of any one of claims 31 to 38, wherein the plurality of acute, fractionated, whole body irradiation treatments comprises 10 irradiation treatments delivered to the subject within 20 days.

41. The use of any one of claims 31 to 40, wherein the radiation is applied at a dose rate of about 0.9 Gy/hr during the irradiation treatments.

42. The use of any one of claims 31 to 41, wherein administering the checkpoint inhibitor to the subject comprises administering a plurality of doses of the checkpoint inhibitor to the subject during the treatment period.

43. The use of claim 42, wherein a number of doses of the checkpoint inhibitor administered to the subject during the treatment period is different than a number of irradiation treatments delivered to the subject during the treatment period.

44. The use of claim 42 or 43, wherein at least two of the irradiation treatments are delivered between consecutive doses of the checkpoint inhibitor

45. The use of claim 42 or 43, wherein each irradiation treatment is delivered to the subject on the same day as one of the doses of the checkpoint inhibitor.

46. The use of claim 45, wherein each irradiation treatment is delivered to the subject between about 30 minutes and about 6 hours after one of the doses of checkpoint inhibitor.

47. The use of any one of claims 42 to 46, wherein each administered dose of the checkpoint inhibitor is between about 5 mg/kg and about 20 mg/kg.

48. The use or use according to claim 47, wherein each administered dose of the checkpoint inhibitor is about 5 mg/kg.

49. The use of any one of claims 31 to 48, wherein a cumulative dosage of the checkpoint inhibitor administered to the subject during the treatment period is about 50 mg/kg.

50. The use of any one of claims 31 to 49, wherein the biologically active amount of the checkpoint inhibitor within the subject is provided by administering doses of between about 5 mg/kg and about 20 mg/kg to the subject at a frequency that is substantially equal to the half-life of the checkpoint inhibitor within the subject. the subject is between about 5 mg/kg and about 20 mg/kg.

51. The use of any one of claims 31 to 50, wherein the treatment period is between 14 and 60 days.

52. The use of any one of claims 31 to 51, wherein the treatment period is between 20 and 40 days.

53. The use of any one of claims 31 to 52, wherein the irradiation is delivered via an irradiation source that comprises cobalt-60.

54. The use of any one of claims 31 to 53, wherein the checkpoint inhibitor is a monoclonal antibody.

55. The use of claim 54, wherein the monoclonal antibody is PD-1.

56. The use of any one of claims 31 to 55, wherein the checkpoint inhibitor inhibits PD-L1, CTLA-4, or a combination thereof.

57. The use of any one of claims 31 to 56, wherein the checkpoint inhibitor interacts with a ligand of PD-L1, CTLA-4, or a combination thereof.

58. The use of any one of claims 31 to 57, wherein the checkpoint inhibitor is administered subcutaneously, intraperitoneally, or intravenously.

59. The use of any one of claims 31 to 58, wherein the tumour is a tumour associated with colon adenocarcinoma.

60. The use of any one of claims 31 to 59, wherein each irradiation treatment is administered to the subject while the biologically active amount of the checkpoint inhibitor is present within the subject.

61. A method of preventing tumour growth in a subject in need thereof comprising administering a checkpoint inhibitor in combination with ultralow dose whole body irradiation to the subject.

62. Use of a checkpoint inhibitor in combination with ultralow dose whole body irradiation in a subject for the prevention of tumour growth.

63. The method or use according to claim 61 or 62, wherein the checkpoint inhibitor is a monoclonal antibody.

64. The method or use according to claims 61-63, wherein the checkpoint inhibitor inhibits PD-L1, CTLA-4, or a combination thereof.

65. The method or use according to claims 61-63, wherein the checkpoint inhibitor interacts with a ligand of PD-L1, CTLA-4, or a combination thereof.

66. The method or use according to claim 65, wherein the monoclonal antibody is PD-1.

67. The method or use according to claims 61-66, wherein the checkpoint inhibitor is administered through a plurality of treatments.

68. The method or use according to claim 67, wherein the checkpoint inhibitor is administered twice a week.

69. The method or use according to claim 67 or 68, wherein the checkpoint inhibitor is administered for five (5) weeks.

70. The method or use according to claims 67-69, wherein each administered dosage of the checkpoint inhibitor is between about 5 mg/kg and about 20 mg/kg.

71. The method or use according to claim 69, wherein each administered dosage of the checkpoint inhibitor is about 5 mg/kg.

72. The method or use according to claims 67-69, wherein the cumulative dosage of the checkpoint inhibitor administered is about 50 mg/kg.

73. The method or use according to claims 61-72, wherein the checkpoint inhibitor is administered subcutaneously, intraperitoneally, or intravenously.

74. The method or use according to claims 61-73, wherein the source of the irradiation is a cobalt-60.

75. The method or use according to claims 1-74, wherein the irradiation is applied at a dose rate of about 0.9 Gy/hour.

76. The method or use according to claims 61-75, wherein the irradiation is administered acutely.

77. The method or use according to claims 61-76, wherein the irradiation is administered through a plurality of treatments.

78. The method or use according to claim 77 wherein the irradiation is administered twice a week.

79. The method or use according to claim 77 or 78, wherein the irradiation is administered for five (5) weeks.

80. The method or use according to claim 77 wherein the irradiation is administered every other day.

81. The method or use according to claim 77 or 80, wherein the irradiation is administered 20 days.

82. The method or use according to claims 61-81, wherein the irradiation is administered at a dose of about 10 mGy and about 100 mGy.

83. The method or use according to claim 82, wherein the irradiation is administered at a dose of about 10 mGy.

84. The method or use according to claims 61-83, wherein the cumulative dosage of irradiation administered is equal to or less than 1 Gy.

85. The method or use according to claims 61-84, wherein the cumulative dosage of irradiation administered is equal to or less than 100 mGy.

86. The method or use according to claims 61-85, wherein the checkpoint inhibitor is administered prior to, at the same time as, or after the irradiation is administered.

87. The method or use according to claims 61-86, wherein the checkpoint inhibitor is administered on the same day as the irradiation.

88. The method or use according to claim 86 or 87, wherein the checkpoint inhibitor is administered at least 30 minutes prior to the administration of the irradiation,

89. The method or use according to claims 61-88, wherein the tumour is a tumour associated with colon adenocarcinoma.
